(19) United States
(12) Patent Application Publication
(10) Pub. No.: US 2012/0071722 A1
(43) Pub. Date: Mar. 22, 2012

(54) ENDOSCOPE AND FLEXIBLE PORTION THEREOF

(75) Inventors: Takayuki NAKAMURA, Ashigarakami-gun (JP); Maki SAITO, Ashigarakami-gun (JP); Atsuhiko ISHIHARA, Ashigarakami-gun (JP); Shozo IYAMA, Ashigarakami-gun (JP)

(73) Assignee: FUJIFILM CORPORATION, Tokyo (JP)

(21) Appl. No.: 13/238,579

(22) Filed: Sep. 21, 2011

(30) Foreign Application Priority Data
Sep. 22, 2010 (JP) 2010-212458

(51) Int. Cl. A61B 1/005

(52) U.S. Cl. 600/140

(57) ABSTRACT

An endoscope comprising:
an endoscope flexible portion that includes:
a low-hardness varying portion which is located in a predetermined region extending from a distal end of the flexible portion, and has hardness varying from the lowest hardness in the flexible portion to a predetermined hardness higher than the lowest hardness, the hardness being the lowest hardness at the distal end of the flexible portion, the hardness being the predetermined hardness at a proximal end of the low-hardness varying portion;
a hard portion which is located in a predetermined region extending from a proximal end of the flexible portion, and has the highest hardness in the flexible portion; and
an intermediate-hardness varying portion which is located between the low-hardness varying portion and the hard portion, and has hardness gradually varying in a region extending from the proximal end of the low-hardness varying portion to a distal end of the hard portion.

### BACKGROUND OF THE INVENTION

1. Field of the Invention

The present invention relates to an endoscope and a flexible portion thereof, and particularly, to an endoscope that enables adjustment of the flexibility of the flexible portion in the insertion unit, and the flexible portion.

2. Description of the Related Art

Conventionally, medical examinations using endoscopes have been widely conducted in the field of medicine. Particularly, an imaging element such as a CCD is built in the distal end of the insertion unit of an endoscope to be inserted into a body cavity, and captures images inside the body cavity. Signal processing is then performed on the images by a processor device, and is displayed on a monitor. A physician observes the images, and uses the images for diagnoses. Alternatively, a treatment tool is inserted through a channel for treatment tool insertion. With such a treatment tool, samples are collected, or polypectomy is performed, for example.

An endoscope is normally formed by connecting a handheld operation unit (a main operation unit) that is held and operated by a practitioner (hereinafter referred to simply as the operator), to an insertion unit that is to be inserted into a body cavity or the like, and by extending a universal cable from the handheld operation unit to a connector unit or the like. The universal cable is extended from the handheld operation unit, and the other end of the universal cable is detachably connected to a light source device (a light source device and a processor).

The insertion unit of the endoscope includes a flexible portion having flexibility so that the insertion unit can be inserted into an insertion path that is bent and curved in a complicated manner. However, because of the flexibility, the distal end of the insertion unit is not stabilized in one direction, and therefore, it is difficult to insert the insertion unit in an intended direction. In some cases, the shape of the insertion unit is preferably kept in conformity with a body cavity, so as to perform some treatment or observation. Therefore, there has been a suggested technique by which a hardness changeable device formed with a coil pipe and a wire is provided in the insertion unit of an endoscope, for example, and an operator operates the hardness changeable device to adjust the flexibility of the insertion unit of the endoscope.

For example, Japanese Patent No. 3869060 discloses an endoscope that has the hardness distribution in the area between the distal end of the flexible tube forming the insertion unit of the endoscope and the handheld side, as shown by graph A in FIG. 14. A predetermined zone extending from the distal end of the flexible tube is a soft flexible portion having the lowest hardness. A predetermined zone on the handheld side is a hard flexible portion having the highest hardness. The zone between the soft flexible portion and the hard flexible portion is a hardness varying zone in which the hardness varies from the lowest hardness to the highest hardness.

In this endoscope, the distal end of the hardness changeable device is located at a predetermined distance from the distal end in the soft flexible portion, and the hardness changeable device is formed along the flexible tube. In this manner, the hardness of the flexible tube can be adjusted to a desired hardness.

FIG. 15 shows how the hardness changeable device changes the hardness distribution in the area between the distal end of the flexible tube and the handheld side. The graph B in FIG. 15 shows the hardness distribution in the flexible tube in a softened state where the hardness changeable device is not operated. This hardness distribution is the same as the hardness distribution obtained in a case where the hardness changeable device is not provided as shown by the graph A in FIG. 14. The graph C in FIG. 15 shows the hardness distribution in the flexible tube when the highest hardness is achieved by operating the hardness changeable device. As shown in FIG. 15, when the hardness changeable device is operated, the hardness of the portion at which the hardness changeable device of the flexible tube is positioned becomes higher by H1 at a maximum.

However, according to the above conventional art, the hardness of the soft flexible portion having a predetermined length from the distal end of the flexible tube is uniform particularly when the hardness changeable device is not operated, as shown by the graph A in FIG. 14 (or by the graph B in FIG. 15). Therefore, when a force is applied to the distal end of the flexible tube, stress is applied only to the boundary (denoted by reference character P on the graph in FIG. 14) between the soft flexible portion and the hardness varying zone. The boundary is the furthest from the point of application. As a result, the curvature of the flexible portion might have an unnatural distribution.

As shown by the graph C in FIG. 15, even when the hardness changeable device is operated to achieve a hardened state, the rigidity in the hardness varying zone is low if the hardness difference between the soft flexible portion and the hardness varying zone is large. When the hardness is increased by the hardness changeable device, a sudden change in hardness occurs at the distal end of the hardness varying zone, and stress is applied to the flexible tube only at the distal end of the hardness varying zone, resulting in a sudden change in curvature.

### SUMMARY OF THE INVENTION

The present invention has been made in view of the above circumstances, and the object thereof is to provide an endoscope that reduces the variation in hardness of the insertion unit so as to prevent sudden bending of the insertion unit, and can achieve an optimum hardness distribution for insertion. The present invention also provides the flexible portion of such an endoscope.

To achieve the above object, a first aspect of the present invention provides an endoscope which includes an endoscope flexible portion including a low-hardness varying portion which is located in a predetermined region extending from the distal end of the flexible portion, and has hardness varying from the lowest hardness in the flexible portion to a predetermined hardness higher than the lowest hardness, the hardness being the lowest hardness at the distal end of the flexible portion, the hardness being the predetermined hardness at the proximal end of the low-hardness varying portion; a hard portion which is located in a predetermined region extending from the proximal end of the flexible portion, and has the highest hardness in the flexible portion; and an intermediate-hardness varying portion which is located between the low-hardness varying portion and the hard portion, and has hardness gradually varying in a region extending from the proximal end of the low-hardness varying portion to a distal end of the hard portion.

In this structure, the flexible portion includes the low-hardness varying portion, the intermediate-hardness varying portion, and the hard portion, and a hardness gradient is caused in each of the low-hardness varying portion and the intermediate-hardness varying portion. Accordingly, an optimum hardness distribution for insertion can be realized.

According to a second aspect of the present invention, the endoscope may further include a hardness adjusting device which changes the hardness of the flexible portion and is located in the flexible portion. The hardness adjusting device includes: a hardness adjusting member which is capable of changing the flexibility of the flexible portion; a hardness changing device which acts on the hardness adjusting member and changes the hardness of the hardness adjusting member; and a drive device which drives the hardness changing device.

Since a hardness gradient is caused in each of the low-hardness varying portion and the intermediate-hardness varying portion of the flexible portion, the variation in hardness of the insertion unit at the point of change in the low-hardness varying portion and at the location of the distal end of the hardness adjusting member can be reduced, and sudden bending of the insertion unit can be prevented, even if the hardness is changed to vary the flexibility of the flexible portion.

According to a third aspect of the present invention, the distal end portion of the hardness adjusting member may be located in the low-hardness varying portion.

According to a fourth aspect of the present invention, the distal end portion of the hardness adjusting member may be located in the intermediate-hardness varying portion.

The distal end of the hardness adjusting member has the above described effect whether the distal end is located in the low-hardness varying portion or in the intermediate-hardness varying portion.

According to a fifth aspect of the present invention, the hardness adjusting member may be a contact spring, the hardness changing device may be a wire which is inserted through the contact spring, and the drive device may be a wire pulling device which pulls the wire.

In this structure, the hardness can be changed by simple tools such as a contact spring and a wire.

According to a sixth aspect of the present invention, the variation in hardness of the low-hardness varying portion and the intermediate-hardness varying portion forming the flexible portion may be formed by causing a gradient in the hardness of the resin layer forming the outer coat of the flexible tube forming the flexible portion.

According to a seventh aspect of the present invention, the hardness gradient in the resin layer may be caused through two-layer molding performed by varying the thickness ratio between a soft resin and a hard resin.

As the resin layer forming the outer coat of the flexible tube forming the flexible portion is formed through two-layer molding, a desired hardness distribution can be readily achieved.

Also, to achieve the above object, an eighth aspect of the present invention provides a flexible portion of an endoscope. The flexible portion includes: a low-hardness varying portion which is located in a predetermined region extending from the distal end of the flexible portion, and has hardness varying from the lowest hardness in the flexible portion to a predetermined hardness higher than the lowest hardness; a hard portion which is located in a predetermined region extending from the proximal end of the flexible portion, and has the highest hardness in the flexible portion; and an intermediate-hardness varying portion which is located between the low-hardness varying portion and the hard portion, and has hardness gradually varying in the region extending from the proximal end of the low-hardness varying portion to the distal end of the hard portion.

In this structure, the flexible portion includes the low-hardness varying portion, the intermediate-hardness varying portion, and the hard portion, and a hardness gradient is caused in each of the low-hardness varying portion and the intermediate-hardness varying portion. Accordingly, an optimum hardness distribution for insertion can be realized.

According to a ninth aspect of the present invention, the flexible portion may further include a hardness adjusting device which includes: a contact spring which has the distal end thereof located in the low-hardness varying portion, and is capable of changing the flexibility of the flexible portion; and a wire which is pulled and relaxed to act on the contact spring and change the hardness of the contact spring.

According to a tenth aspect of the present invention, the flexible portion may further include a hardness adjusting device which includes: a contact spring which has the distal end thereof located in the intermediate-hardness varying portion, and is capable of changing the flexibility of the flexible portion; and a wire which is pulled and relaxed to act on the contact spring and change the hardness of the contact spring.

With this arrangement, the variation in hardness of the insertion unit at the point of change in the low-hardness varying portion and at the location of the distal end of the contact spring of hardness adjusting member can be reduced, and sudden bending of the insertion unit can be prevented.

As described above, according to the present invention, the flexible portion includes the low-hardness varying portion, the intermediate-hardness varying portion, and the hard portion, and an optimum hardness distribution for insertion can be realized by causing a hardness gradient in each of the low-hardness varying portion and the intermediate-hardness varying portion. Also, in a case where the hardness adjusting device is provided in the flexible portion, the variation in hardness of the insertion unit at the point of change in the low-hardness varying portion and at the location of the distal end of the hardness adjusting member can be reduced, and sudden bending of the insertion unit can be prevented, even if the hardness is changed to vary the flexibility of the flexible portion. An optimum hardness distribution for insertion can also be realized in such a case.

### BRIEF DESCRIPTION OF THE DRAWINGS

FIG. 1 is a schematic view showing the structure of an embodiment of an endoscope according to the present invention;

FIG. 2 is a longitudinal cross-sectional view of the internal structure of the endoscope;

FIG. 3 is a cross-sectional view of the handheld operation unit, showing the structure of the wire pulling unit;

FIG. 4 is a perspective view showing the structure of the wire pulling unit;

FIG. 5 is a diagram showing the hardness distribution of the flexible portion of this embodiment;

FIG. 6 is a diagram showing a comparison between the hardness distribution in a case where the hardness adjusting device of the flexible portion of this embodiment is activated, and the hardness distribution in a case where the hardness adjusting device is not activated;

FIG. 7 is a partial cross-sectional view schematically showing the structure of the flexible tube forming the flexible portion;

FIG. 8 is a block diagram schematically showing the structure of an apparatus for manufacturing the flexible tubes of endoscopes;

FIG. 9 is a cross-sectional view showing the relevant components in the structure of the head unit;

FIG. 10 is a cross-sectional view of the head unit, taken along the line A-A of FIG. 9;

FIG. 11A is a diagram showing a comparative example in which the difference in melt viscosity at a molding temperature is large;

FIG. 11B is a diagram showing this embodiment in which the difference in melt viscosity is small;

FIG. 12 is a graph showing the hardness distributions in the axial direction of flexible tubes having different 100% modulus values;

FIG. 13 is a diagram for explaining a method of measuring the hardness distribution of a flexible tube;

FIG. 14 is a diagram showing the hardness distribution of a conventional flexible tube; and

FIG. 15 is a diagram showing a comparison between the hardness distribution in a case where a hardness changing device of the conventional flexible tube is activated and the hardness distribution in a case where the hardness changing device is not activated.

### DETAILED DESCRIPTION OF THE PREFERRED EMBODIMENTS

The following is a detailed description of an endoscope and the flexible portion of the endoscope according to the present invention, with reference to the accompanying drawings.

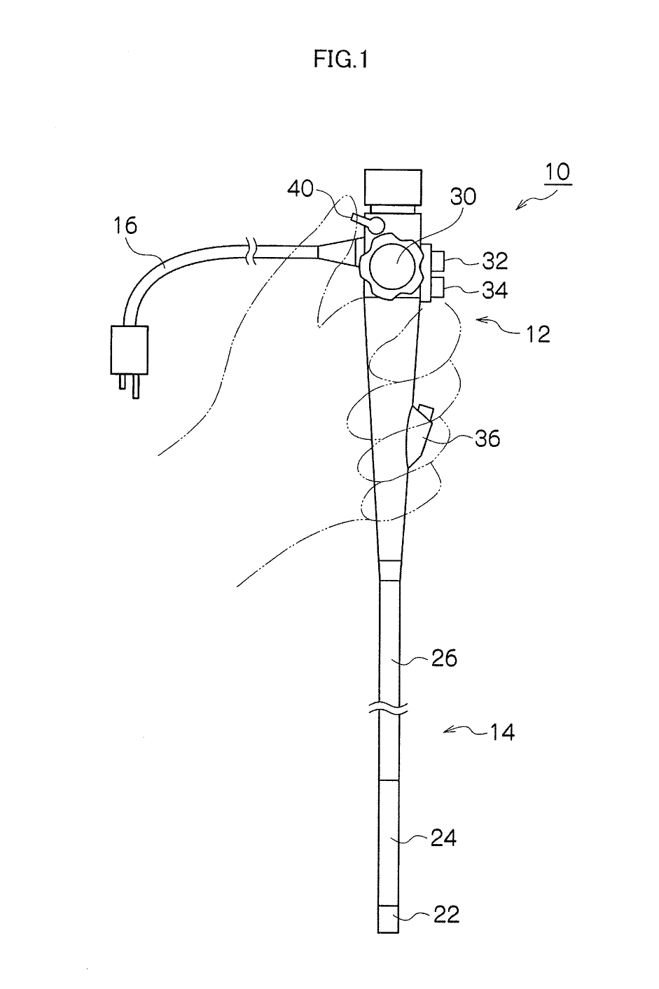

FIG. 1 is a schematic view showing the structure of an embodiment of an endoscope according to the present invention.

As shown in FIG. 1, the endoscope 10 of this embodiment includes a handheld operation unit 12 and an insertion unit 14 joined to the handheld operation unit 12. An operator holds and handles the handheld operation unit 12 with his/her left hand as indicated by the double-dot dash line in the drawing, and holds the insertion unit 14 with his/her right hand (not shown). While doing so, the operator inserts the insertion unit 14 into a body cavity of a subject, and conducts an observation.

A universal cable 16 is connected to the handheld operation unit 12. Although not shown in the drawing, a LG connector is attached to the distal end of the universal cable 16, and the LG connector is detachably connected to a light source device. With this arrangement, illumination light is supplied to an illumination optical system provided at the distal end portion of the insertion unit 14. Also, though not shown in the drawing, an electric connector is connected to the LG connector via the universal cable 16, and the electric connector is detachably connected to an endoscope processor. With this arrangement, the data about an observed image obtained by the endoscope 10 is output to the endoscope processor, and the image is displayed on a monitor device connected to the endoscope processor. Using the displayed image, an operator conducts an observation.

The insertion unit 14 is connected to the distal end of the handheld operation unit 12 as shown in FIG. 1. The insertion unit 14 includes a flexible portion 26, a curving portion (an angled portion) 24, and a distal end portion 22, when seen from the proximal end (on the side of the handheld operation unit 12) toward the distal end (on the side to be inserted into a body cavity). The curving portion 24 is remotely handled to curve when an angled knob 30 provided on the handheld operation unit 12 is rotated. Accordingly, the distal end surface of the distal end portion 22 can be made to face in a desired direction.

The handheld operation unit 12 includes: an air/water supply button 32 for supplying air and water from an air/water supply outlet at the distal end portion 22 to an area to be examined or the like via an air/water supply channel; a suction button 34 for applying suction from a forceps slit at the distal end portion 22 via a forceps channel; and a forceps insertion slot 36 that is an opening continuing to the forceps channel and is designed for an operator to insert forceps therethrough.

The endoscope 10 also includes a hardness adjuster that adjusts the hardness (or changes the flexural toughness or flexibility) of the flexible portion 26. As will be described later in detail, a contact spring (a coil) is provided in the flexible portion 26, and a wire is firmly fixed to the contact spring on the distal end side of the flexible portion 26 and is inserted through the contact spring fixed to a fixing member on the side of the handheld operation unit 12. The wire is pulled to compress the contact spring and increase the hardness of the contact spring. In this manner, the hardness of the flexible portion 26 is increased.

A handle lever 40 of a hardness adjusting device to adjust the hardness of the flexible portion 26 is provided at the upper portion of the handheld operation unit 12. When the handle lever 40 is operated, the wire is pulled via a wire pulling unit. Particularly, the handle lever 40 is positioned within such a region that the thumb of the left hand holding the handheld operation unit 12 can reach the handle lever 40, as indicated by the double-dot dash line in FIG. 1.

Further, as will be described later in detail, the wire pulling unit of the hardness adjusting device and the fixing member for the contact spring subjected to the wire pulling force are also provided in the upper portion of the handheld operation unit 12 in this embodiment.

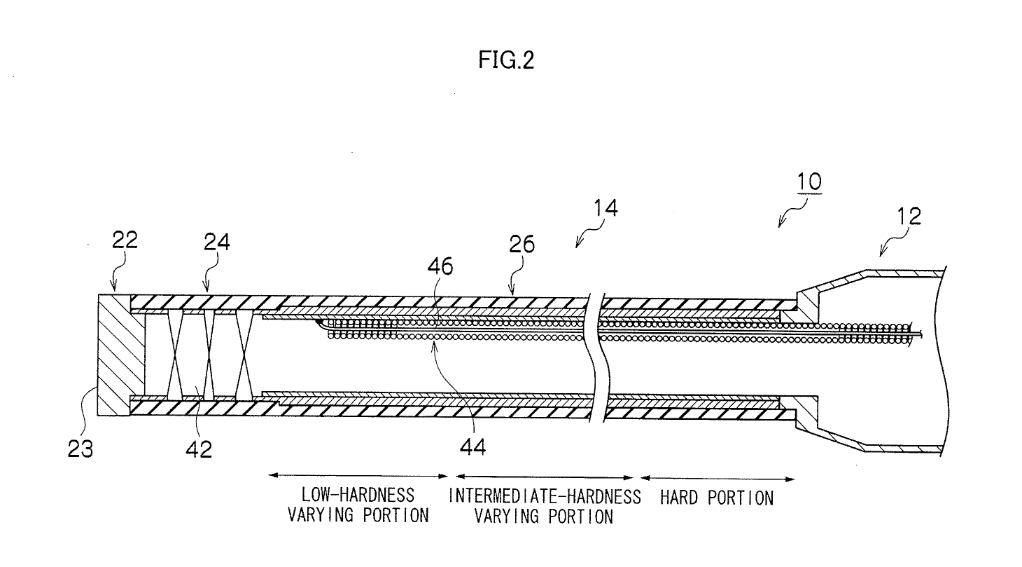

FIG. 2 is a longitudinal cross-sectional view of the structure of the endoscope 10.

As shown in FIG. 2, the curving portion 24 of the insertion unit 14 is formed by a large number of curving pieces 42 (angled members) each having a ring-like shape. Each two curving pieces 42 are rotatably joined to each other. When the angled knob 30 (see FIG. 1) of the handheld operation unit 12 is turned, the curving portion 24 is curved horizontally or vertically. Accordingly, the distal end surface 23 of the distal end portion 22 can be made to face in a desired direction.

Also, as shown in FIG. 2, the flexible portion 26 includes a low-hardness varying portion, an intermediate-hardness varying portion, and a hard portion, which will be described later. Further, a contact spring (a hardness adjusting coil) 44 forming the hardness adjusting device, and a wire (a hardness adjusting wire) 46 inserted through the contact spring 44 are provided inside the flexible portion 26.

The wire 46 inserted through the contact spring 44 has one end fixed to the distal end of the contact spring 44, and has the other end connected to the wire pulling unit that is placed inside the handheld operation unit 12 but is not shown in FIG. 2. As described above, when the handle lever 40 provided at the upper portion of the handheld operation unit 12 is operated, the wire 46 is pulled by the wire pulling unit. As a result, the contact spring 44 is compressed, and is put into a hardened state with low flexibility. In this manner, the hardness of the flexible portion 26 is adjusted to become higher.

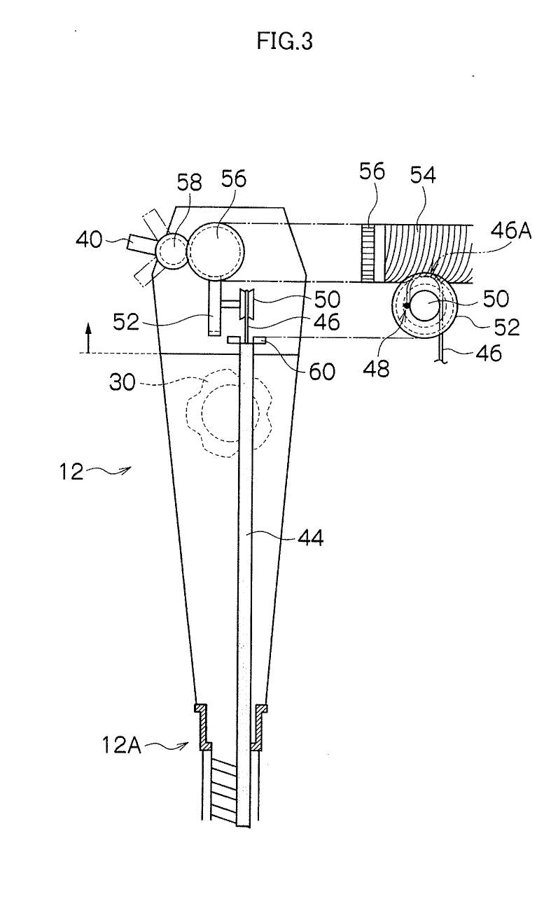

FIG. 3 illustrates the structure of the wire pulling unit. The left half of FIG. 3 is a cross-sectional view of the handheld operation unit 12, and the right half of FIG. 3 is a side view of the wire pulling mechanism seen from the right side of the handheld operation unit 12 in the drawing.

As shown in the left half of FIG. 3, a wire pulley 50 of the wire pulling unit for pulling the wire 46 inserted through the contact spring 44 is provided in the upper portion of the handheld operation unit 12.

The wire 46 is wound around the wire pulley 50. The wire pulley 50 is coaxially connected to a worm wheel (a pulley drive gear) 52.

As shown in the right half of FIG. 3, the worm wheel 52 meshes with a worm 54. A spur gear 56 is coaxially connected to the worm 54, and the spur gear 56 meshes with a gear 58 connected to the handle lever 40. The worm wheel 52 and the worm 54 form a worm gear (a speed reduction mechanism). It should be noted that the speed reduction mechanism is not limited to a structure formed by gears, but may be a mechanism formed by a chain and a belt.

The wire 46 has its end point 48 fixed to the wire pulley 50. Also, the fixing member 60 that fixes the contact spring 44 is provided in the immediate vicinity of the wire pulley 50 (a wire pulling device) provided in the upper portion of the handheld operation unit 12.

The variable hardness adjusting unit, the wire pulling unit, and the contact spring fixing unit are collectively positioned in the upper portion of the handheld operation unit, so that the portion of the handheld operation unit above the contact spring fixing member is regarded as a function enhanced module that is an independent module, for example, as indicated by the up-arrow in FIG. 3. With this arrangement, maintenance becomes easier than in a case where those units are positioned between the handheld operation unit and the flexible portion.

When an operator operates the handle lever 40, the gear 58 connected to the handle lever 40 is driven, and the spur gear 56 is driven accordingly. As a result, the worm 54 coaxially connected to the spur gear 56 is driven. The worm wheel 52 is then driven by the worm 54, and the wire pulley 50 is rotated to pull the wire 46.

The distal end of the wire 46 is fixed to the distal end of the contact spring 44, and one end of the contact spring 44 is fixed to the fixing member 60. Therefore, when the wire 46 is pulled, the contact spring 44 is pulled toward the wire pulley 50 of the wire pulling unit, and is compressed with the fixing member 60. Accordingly, the hardness of the contact spring 44 becomes higher.

As described above, in this embodiment, the fixing member 60 that fixes the contact spring 44 is provided in the upper portion of the handheld operation unit 12, to extend the contact spring 44 to the upper portion of the handheld operation unit 12.

Also, the handle lever 40 is designed to be moved up and down, as shown in FIG. 3. When the handle lever 40 is moved upward, the spur gear 56 is driven by the gear 58, the worm 54 is driven with the spur gear 56, and the worm wheel 52 is driven by the worm 54. As a result, the wire pulley 50 is rotated in the wind-up direction of the wire 46, and the wire 46 is pulled to compress the contact spring 44. The hardness of the contact spring 44 then becomes higher, and the hardness of the flexible portion 26 also becomes higher (or the flexibility of the flexible portion 26 becomes lower). When the handle lever 40 is moved downward, the respective gears are driven in the opposite directions of the above, and the wire pulley 50 is rotated in the wind-down direction of the wire 46. The wire 46 is then relaxed, and the contact spring 44 is expanded. Accordingly, the hardness of the contact spring 44 becomes lower, and the hardness of the flexible portion 26 also becomes lower (or the flexibility of the flexible portion 26 becomes higher).

Here, the operation force is transmitted from the handle lever 40 to the worm 54 through the gear 58 of the handle lever 40, and is further transmitted to the wire pulley 50 through the worm wheel 52. However, the wire 46 is fixed to the distal end of the contact spring 44, and the contact spring 44 is curved and becomes longer when the insertion unit 14 (the flexible portion 26) is curved. Therefore, even when the handle lever 40 is not operated, the wire 46 is pulled toward the wire pulley 50 in a relative manner, and the hardness of the contact spring 44 varies. To prevent the hardness of the contact spring 44 from varying when the insertion unit 14 is curved while the handle lever 40 is not operated and the hardness is zero, the wire 46 has initial slack (an initial extra length) as indicated by the reference numeral 46A in FIG. 3.

When an operator operates the handle lever 40 to increase the hardness of the flexible portion 26, the worm wheel 52 is secured in the current position by the friction between the gear tooth surfaces of the worm 54 and the worm wheel 52 even if the operator takes his/her thumb off the handle lever 40. As the worm wheel 52 is secured by the worm 54 in this manner, the wire pulley 50 is secured in a desired position, and the wire 46 can be maintained in a pulled state. As described above, the worm gear formed by the worm wheel 52 and the worm 54 has the braking function (a self-lock function) to hold the wire pulled state. The worm gear also has a speed reducing function, and is incorporated into the structure to reduce the wire pulling force that acts on the wire 46 and reaches several tens of kilograms of force (kgf) to a smaller operation force.

As described above, according to this embodiment, the wire 46 is pulled to increase the hardness of the contact spring 44. However, the distal end of the wire 46 is tightly bonded to the contact spring 44 on the distal end side of the flexible portion 26. Therefore, the pulling force for the wire 46 acts, as the compression force for the contact spring 44, on the fixing member 60 for the contact spring 44 located in the upper portion of the handheld operation unit 12. That is, the wire pulling mechanism and the fixing member 60 for the contact spring 44 are structurally connected to each other, to keep the equilibrium of force.

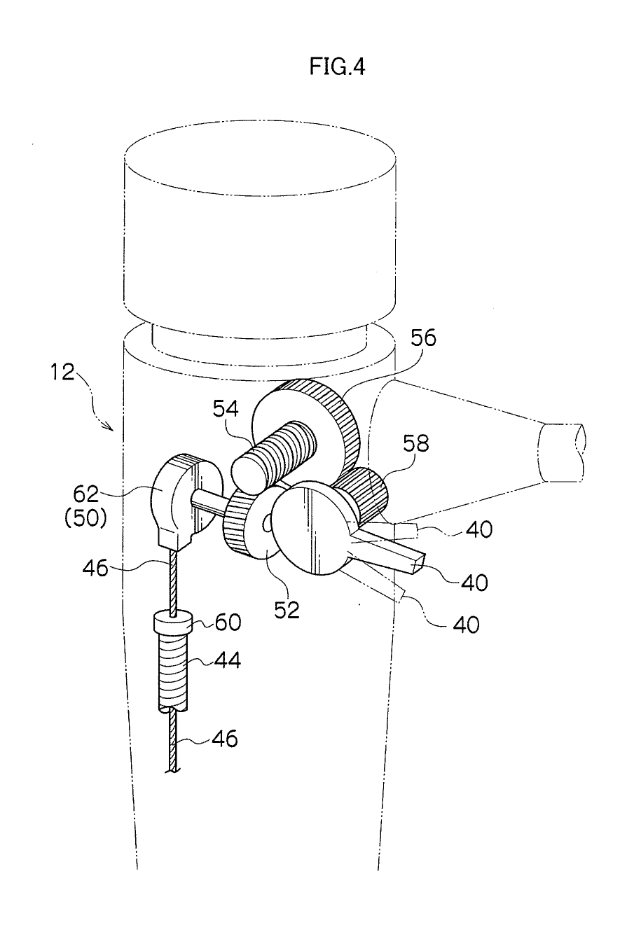

FIG. 4 is a perspective view of the wire pulling mechanism shown in a simplified manner. Referring to this drawing, the wire pulling mechanism is again described (though some of the above explanation will be repeated).

As shown in FIG. 4, the pulling mechanism unit includes the fixing member 60 for the contact spring 44, a pulley housing 62 housing the pulley 50, the worm wheel 52, the worm 54, the spur gear 56, and the gear 58, when seen from the wire 46 toward the handle lever 40.

In FIG. 4, the other end of the contact spring 44 is fixed to the contact spring fixing member 60 by brazing or the like. The end portion of the wire 46 is inserted through the fixing member 60 for the contact spring 44, and is connected to the pulley 50 in the pulley housing 62.

The pulley 50 in the pulley housing 62 is coaxially connected to the worm wheel 52, and the worm wheel 52 meshes with the worm 54. The spur gear 56 is coaxially connected to the worm 54, and the gear 58 that is coaxially connected to the handle lever 40 meshes with the spur gear 56.

The helical angle of the worm 54 is smaller than the angle of repose (the angle of friction). Accordingly, reverse driving of the worm 54 by the worm wheel 52 is prevented, and a self-braking force is supplied to the pulley 50 in the pulley housing 62. Further, the speed reduction ratio of the speed reduction mechanism is set at 50:1, for example, so that a torque that is 50 times as large as the operation force of the handle lever 40 is transmitted to the pulley 50. With this arrangement, the wire pulling force that reaches several tens of kilograms can be reduced to a smaller operation force, and the handle lever 40 can be easily handled with a thumb.

That is, with the pulling mechanism of this embodiment, the pulley 50 can be driven through repetitive rotating operations with short strokes of the handle lever 40. In the pulling mechanism, the pulley 50 is rotated through the speed reduction mechanism. Therefore, the operation of the handle lever 40 increases, but the torque corresponding to the speed reduction ratio can be obtained. Accordingly, the pulling force for the wire 46 can be made smaller. Thus, the handle lever 40 can be easily operated with a thumb of an operator. Also, with the speed reduction mechanism, the mechanism unit does not become larger than a pulling mechanism unit that pulls a wire through a rotation caused by one stroke of a handle lever. Also, the handle lever 40 can be small in size. Therefore, the pulling mechanism unit can be made smaller in size. Furthermore, by the self-braking force of the worm gear, the pulled wire 46 can be maintained in a state in which the wire 46 is wound around the pulley 50. Accordingly, the flexibility of the flexible portion 26 can be easily maintained.

When the handle lever 40 is rotationally handled by an operator, the gear 58 connected to the handle lever 40 is driven, and the spur gear 56 is driven accordingly. As a result, the worm 54 and the worm wheel 52 are driven, and the pulley 50 rotates to pull or relax the wire 46. The distal end of the wire 46 is fixed to the distal end of the contact spring 44 as shown in FIG. 2, and the other end of the contact spring 44 is fixed to the fixing member 60 for the contact spring 44. Therefore, when the wire 46 is pulled, the contact spring 44 is pulled toward the pulley 50, and is compressed between the pulley 50 and the contact spring fixing member 60. In this manner, the hardness of the contact spring 44 becomes higher.

When the handle lever 40 is moved upward, the pulley 50 is rotated in such a direction as to wind up the wire 46 through the gear 58, the spur gear 56, the worm 54, and the worm wheel 52. With this, the wire 46 is pulled, and the contact spring 44 is compressed. Accordingly, the hardness of the contact spring 44 becomes higher, and the flexibility of the flexible portion 26 becomes lower (or the flexible portion 26 becomes harder to be bent).

When the handle lever 40 is moved downward, the pulley 50 is rotated in such a direction as to wind down the wire 46 through the gear 58, the spur gear 56, the worm 54, and the worm wheel 52. With this, the wire 46 is relaxed, and the contact spring 44 is released. Accordingly, the hardness of the contact spring 44 becomes lower, and the flexibility of the flexible portion 26 becomes higher (or the flexible portion 26 becomes easier to be bent).

When an operator operates the handle lever 40 to increase the hardness of the flexible portion 26, the worm wheel 52 is secured (self-locked) in the current position by the friction between the gear tooth surfaces of the worm wheel 52 and the worm 54 or by the self-braking force, even if the operator takes his/her thumb off the handle lever 40. As rotational movement of the worm wheel 52 is restrained in this manner, the wire pulley 50 can be secured in a desired position, and the wire 46 can be maintained in a pulled state.

As described above, in the endoscope 10 of this embodiment, the pulling mechanism unit is formed by the pulley 50 that pulls and relaxes the wire 46, and the speed reduction mechanism having the worm gear to supply a self-braking force as well as a rotational drive force to the pulley 50. Accordingly, the pulling mechanism unit can be made smaller in size, and the pulling operation force required for the wire 46 can be made smaller.

In this embodiment, the flexible portion (equivalent to the flexible tube of the above described conventional art) includes a low-hardness varying portion, an intermediate-hardness varying portion, and a hard portion in this order from the distal end to the proximal end, so that a rapid change in hardness is prevented, and an optimum hardness distribution in the insertion unit can be obtained at the time of insertion.

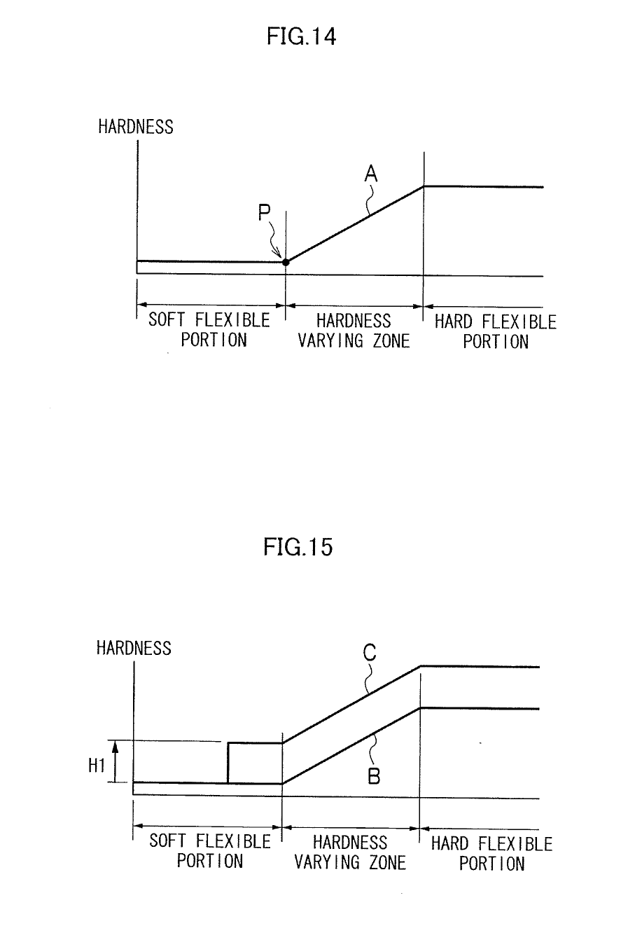

FIG. 5 shows a hardness distribution in the flexible portion 26 from the distal end to the proximal end in this embodiment. As shown in FIG. 2, the flexible portion 26 includes a low-hardness varying portion, an intermediate-hardness varying portion, and a hard portion. The graph D in FIG. 5 indicates the hardness distribution in the low-hardness varying portion, the intermediate-hardness varying portion, and the hard portion of the flexible portion 26. For a comparison purpose, the broken line in FIG. 5 indicates the hardness distribution in a conventional flexible tube (see the graph A of FIG. 14).

As shown in FIG. 5, in the conventional example, the hardness in the portion equivalent to the low-hardness varying portion on the distal end side is uniform, and the hardness suddenly changes at the point denoted by reference character “P”. Therefore, stress concentrates on this point of change. To avoid such a situation in this embodiment, the hardness in the low-hardness varying portion on the distal end side is not uniform but is made to have a gradient, so that the hardness becomes gradually higher in the low-hardness varying portion from the distal end side toward the rear end on the side of the intermediate-hardness varying portion. In the intermediate-hardness varying portion, the hardness becomes gradually higher from the rear end of the low-hardness varying portion toward the distal end of the hard portion.

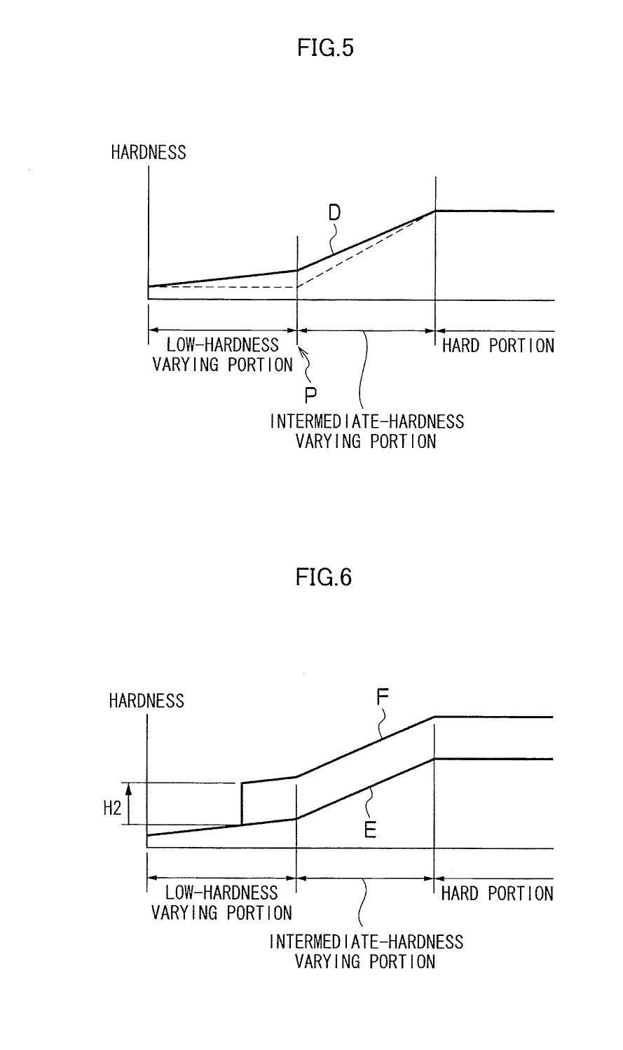

FIG. 6 shows graph E that indicates the hardness distribution in a soft state in which the hardness adjusting device (a hardness changeable member) provided in the flexible portion 26 is not activated, and graph F that indicates the hardness distribution in a hard state in which the hardness adjusting device (the hardness changeable member) is activated.

As shown in FIG. 2, the hardness adjusting device (the contact spring (the hardness adjusting coil) 44) has its distal end located at a predetermined distance from the distal end of the low-hardness varying portion in the low-hardness varying portion on the distal end side of the flexible portion 26. It should be noted that the position at which the distal end of the hardness adjusting device (the contact spring 44) is located is not limited to the position at the predetermined distance from the distal end in the low-hardness varying portion, but may be at the distal end of the low-hardness varying portion, for example. Alternatively, the distal end of the hardness adjusting device (the contact spring 44) may be placed in the intermediate-hardness varying portion.

When the hardness of the contact spring 44 is made higher by operating the handle lever 40 to activate the hardness adjusting device, the hardness distribution in the flexible portion 26 changes as shown by the graph F in FIG. 6. The hardness (the flexural roughness) becomes higher by H2 at a maximum, compared with the case where the hardness adjusting device is not activated.

In this embodiment, the flexible portion 26 is made to have the hardness distribution shown in FIG. 5, so that the difference in hardness between the hardness adjusting device and the low-hardness varying portion becomes smaller. Accordingly, when the endoscope 10 is inserted into a body cavity, a rapid change in rigidity is restrained while the softness required for the insertion is maintained in the distal end region of the flexible portion 26.

With this arrangement, the variation in hardness in the insertion unit can be reduced at the point of change of the hardness distribution in the flexible portion 26 and the distal end position of the hardness adjusting device (the hardness changeable member), and sudden bending can be prevented at the time of insertion.

To form the hardness distribution shown in FIG. 5 in the flexible portion 26, the resin forming the outer coat of the flexible tube forming the flexible 26 should have the two layers of a soft resin and a hard resin. In the two-layer resin molding, the thickness ratio between the two layers is gradually changed so that the hardness gradually changes.

Next, a method of forming the resin outer coat having the hardness distribution indicated by the graph D in FIG. 5 through two-layer molding is described.

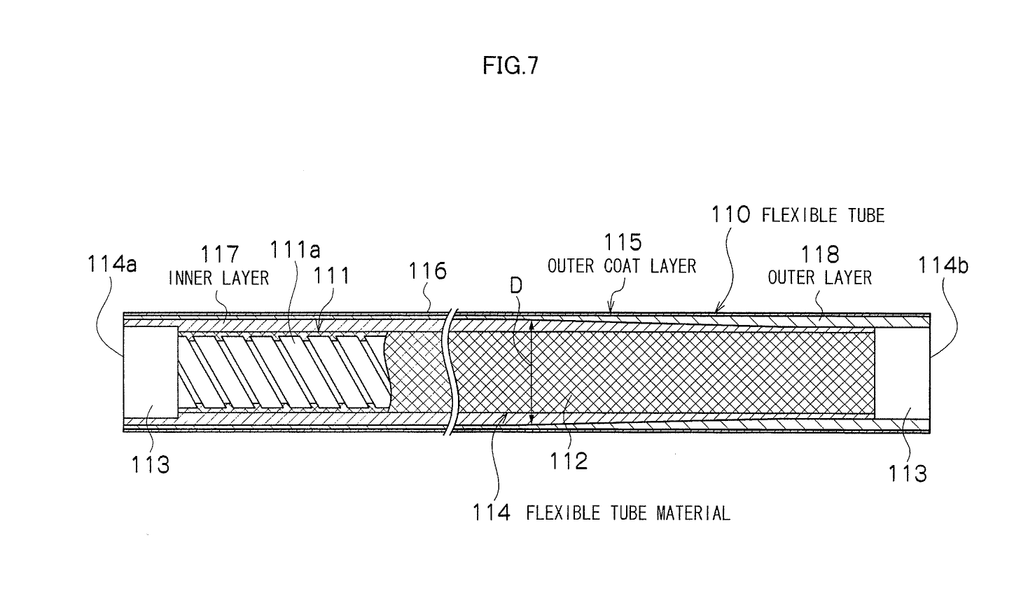

FIG. 7 is a partial cross-sectional view schematically showing the structure of the flexible tube 110 (a flexible tube for an endoscope) forming the flexible portion 26.

As shown in FIG. 7, a spiral tube 111 formed by winding a metal ribbon 111a around the innermost portion thereof in a spiral manner is coated with a cylindrical net 112 formed by braiding a metal wire. Ferrules 113 are engaged with both ends of the spiral tube 111, to form a flexible tube material 114. The outer circumference of the flexible tube material 114 is further coated with an outer coat layer 115 made of a resin. The outer surface of the outer coat layer 115 is also coated with a coat film 116 containing a chemical-resistant substance such as fluorine. Although the spiral tube 111 has only one layer in the drawing, the spiral tube 111 may be formed by coaxially stacking two layers. It should be noted that, to clearly show the layer structure, the thicknesses of the outer coat layer 115 and the coat film 116 are greater in the drawing than in reality in relation to the diameter of the flexible tube material 114.

The outer coat layer 115 coats the outer circumference of the flexible tube material 114. The outer coat layer 115 has a two-layered structure formed by stacking an inner layer 117 coating the entire axial circumference of the flexible tube material 114, and an outer layer 118 coating the entire axial circumference of the inner layer 117. The material of the inner layer 117 is a soft resin, and the material of the outer layer 118 is a hard resin.

The outer coat layer 115 is formed to have a substantially uniform thickness in the longitudinal direction (the axial direction) of the flexible tube material 114. The thickness of the outer coat layer 115 is 0.2 to 1.0 mm, for example, and the outer diameter D of the flexible tube 110 is 11 to 14 mm, for example.

The inner layer 117 and the outer layer 118 are designed so that the thickness ratio between the respective layers 117 and 118 varies with the entire thickness of the outer coat layer 115 in the axial direction of the flexible tube material 114. Specifically, at one end 114a (the distal end) of the flexible tube material 114 attached to the curving portion (the angled portion) 24, the inner layer 117 is thicker than the outer layer 118 with respect to the entire thickness of the outer coat layer 115. The thickness of the inner layer 117 gradually becomes smaller from the one end 114a toward the other end 114b (the proximal end) attached to the handheld operation unit 12. At the other end 114b, the outer layer 118 is thicker than the inner layer 117.

At both ends 114a and 114b, the thickness ratio between the inner layer 117 and the outer layer 118 is the highest. At the one end 114a, the thickness ratio is 9:1, and, at the other end 114b, the thickness ratio is 1:9. The thickness ratio between the inner layer 117 and the outer layer 118 is inverted between the two ends 114a and 114b. Therefore, in the flexible tube 110, there is a hardness difference between the one end 114a and the other end 114b, and the flexibility varies in the axial direction so that the one end 114a becomes softer, and the other end 114b becomes harder.

The thickness ratio between the inner layer 117 and the outer layer 118 is preferably in the range of 1:9 to 9:1 as in the above example. If the ratio is outside the range (0.5:9.5, for example), it is difficult to control the extrusion of the thinner resin, and unevenness easily appears in shape.

Between the soft resin and the hard resin used as the inner layer 117 and the outer layer 118, the difference in 100% modulus value, which is the indicator of the hardness after molding, is 10 MPa or larger, and the difference in melt viscosity, which is the indicator of the fluidity of a resin in a molten state, is 2500 PaS or smaller at a molding temperature of 150 to 200° C., as will be described later. Two such resins are used as the inner layer 117 and the outer layer 118. Accordingly, in the outer coat layer 115 formed by the inner layer 117 and the outer layer 118, excellent molding precision and the hardness difference required between the distal end and the proximal end can be secured.

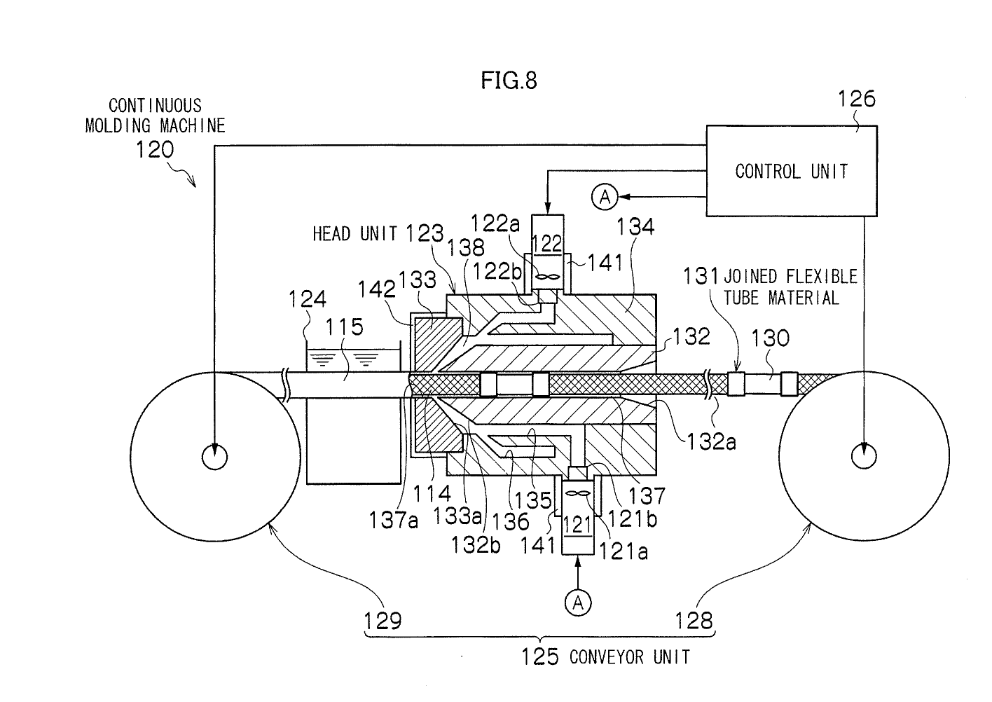

In the following, a method of manufacturing the flexible tube 110 (a method of molding the outer coat layer 115) is first described. FIG. 8 shows the structure of a continuous molding machine 120 that forms the outer coat layer 115. In FIG. 8, the continuous molding machine 120 includes known extrusion units 121 and 122 formed by a hopper and screw 121a and 122a and the like, a head unit 123 for molding the outer coat layer 115 to coat the outer circumference of the flexible tube material 114, a cooling unit 124, a conveyor unit 125 that conveys a joined flexible tube material 131 to the head unit 123, and a control unit 126 that controls those components.

The conveyor unit 125 includes a feeding drum 128 and a winding drum 129. The joined flexible tube material 131 in which flexible tube materials 114 are joined to one another by joint members 130 is wound around the feeding drum 128. After wound around the feeding drum 128, the joined flexible tube material 131 is pulled out, and passes through the head unit 123 at which the outer coat layer 115 is molded, and the cooling unit 124 at which the molded outer coat layer 115 is cooled. The joined flexible tube material 131 is then wound around the winding drum 129. The rotating speeds of the feeding drum 128 and the winding drum 129 are controlled by the control unit 126, so as to change the conveying speed at which the joined flexible tube material 131 is being conveyed.

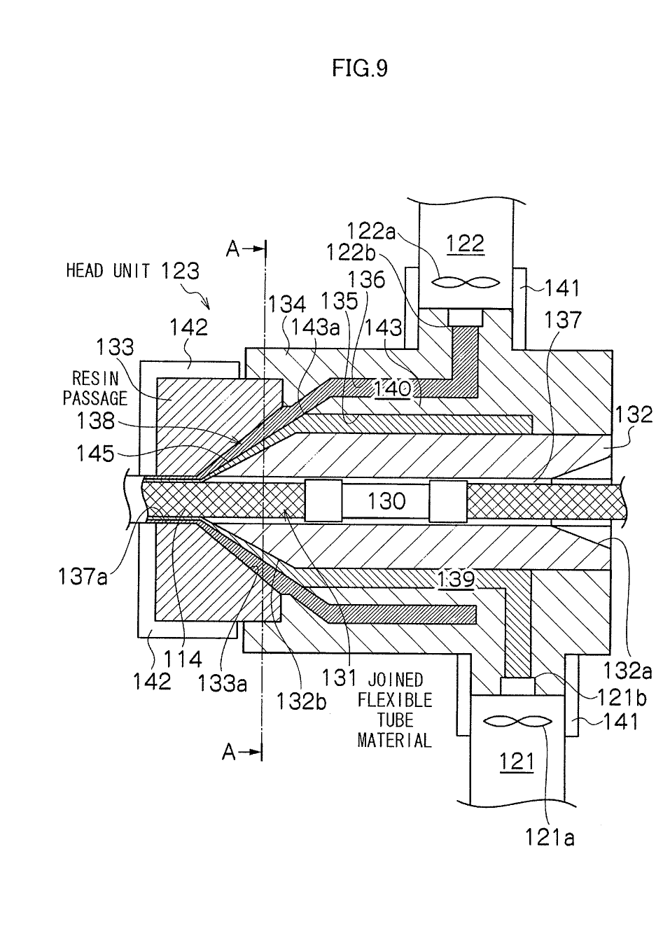

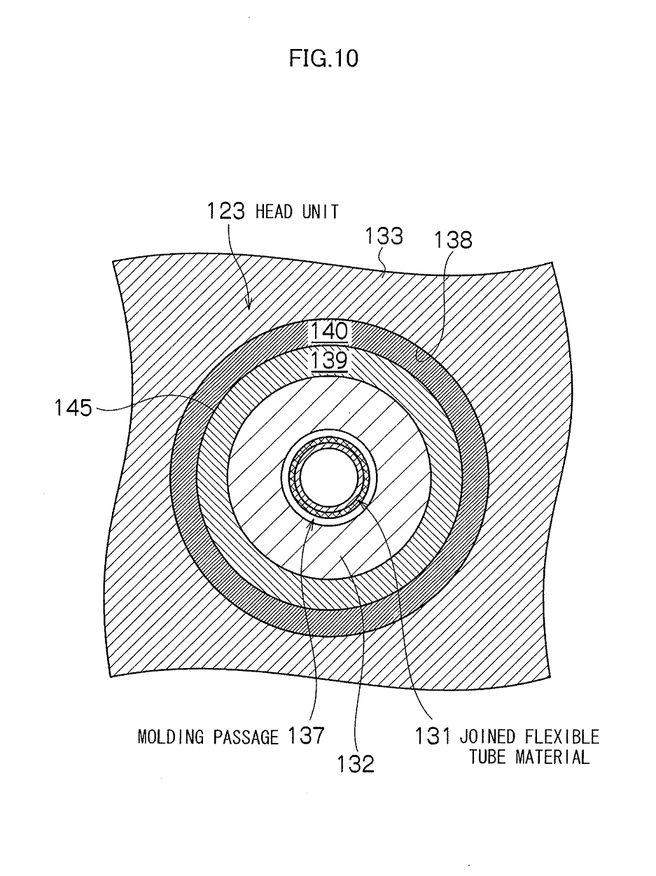

As shown in FIGS. 8 and 9, the head unit 123 includes a nipple 132, a die 133, and a supporting body 134 that firmly supports the nipple 132 and the die 133. In the supporting body 134, gates 135 and 136 are formed to send to the resin passage 138 a soft resin 139 and a hard resin 140 (see also FIG. 10) that are extruded from the extrusion units 121 and 122, respectively, and are in a molten state.

In the nipple 132 and the die 133 serving as a molding tool, a molding passage 137 is formed to penetrate the center portions of the respective components. The molding passage 137 is the passage through which the joined flexible tube material 131 is conveyed in the axial direction by the conveyor unit 125, and has a circular cross-sectional shape in a direction perpendicular to the axial direction (see FIG. 10). The molding passage 137 is connected to a discharge outlet equivalent to the downstream end of the resin passage 138, and the soft resin 139 and the hard resin 140 in a molten state are supplied from the resin passage 138 to the molding passage 137.

The resin passage 138 is formed by the space interposed between the nipple 132 and the die 133. At the left end of the nipple 132 in the drawings, a conical convex portion 132b that forms the resin passage 138 with a conical concave portion 133a at the right end of the die 133 is formed. Also, a conical concave portion 132a that continues to the right end of the molding passage 137 in the drawings, and facilitates insertion of the joined flexible tube material 131 into the molding passage 137 is formed.

The outlet hole 137a of the molding passage 137 is formed in the die 133. The joined flexible tube material 131 having the outer coat layer 115 formed thereon is conveyed to the cooling unit 124 through the outlet hole 137a. A coolant such as water is stored in the cooling unit 124. Passing through the coolant, the outer coat layer 115 is cooled and hardened. Alternatively, the outer coat layer 115 may be cooled by blowing a coolant or air thereonto.

The resin passage 138 is located outside the molding passage 137. The cross-sectional shape of the resin passage 138 in the direction perpendicular to the axial direction of the molding passage 137 is a circle that forms a concentric pattern with the molding passage 137. The discharge outlet of the resin passage 138 connects to the whole circumference of the molding passage 137 in a circumferential direction. Therefore, the soft resin 139 and the hard resin 140 in a molten state are discharged toward the whole circumference of the joined flexible tube material 131 passing through the discharge outlet of the resin passage 138.

The extrusion units 121 and 122 have discharge outlets 121b and 122b connected to the gates 135 and 136 of the head unit 123, respectively. The extrusion units 121 and 122 extrude and supply the molten soft resin 139 and the molten hard resin 140, which are to be the materials of the inner layer 117 and the outer layer 118, into the molding passage 137 in the head unit 123 through the resin passage 138. The number of rotations of each of the screws 121a and 122a is controlled by the control unit 126, so that the amounts of the molten soft resin 139 and the molten hard resin 140 discharged from the extrusion units 121 and 122 are adjusted.

Heating units 141 and 142 are provided in the extrusion units 121 and 122 and in the die 133. The heating units 141 are designed to partially surround the extrusion units 121 and 122 and the gates 135 and 136. The heating units 141 are heaters formed by electrically-heated wires, and are provided in the respective extrusion units 121 and 122. The soft resin 139 and the hard resin 140 extruded from the extrusion units 121 and 122 are heated by the respective heating units 141, so as to have appropriate melt viscosities. The heated soft resin 139 and the hard resin 140 are put into a molten state, and are then sent into the resin passage 138.

The heating unit 142 is designed to surround the outer circumference and the distal end surface of the die 133. Like the heating units 141, the heating unit 142 is a heater formed by an electrically-heated wire, and heats the inside of the die 133 or the insides of the molding passage 137 and the resin passage 138 to a predetermined molding temperature. The molding temperature is set in the range of 150 to 200° C. The soft resin 139 and the hard resin 140 are sent into the resin passage 138 heated to the above molding temperature, and is fed into the molding passage 137 through the resin passage 138.

As the heating units 141 and 142 perform heating and carry out temperature adjustments, the temperatures of the soft resin 139 and the hard resin 140 are set at high temperatures. In addition to that, as the number of rotations of each of the screws 121a and 122a becomes larger, the temperatures of the soft resin 139 and the hard resin 140 become even higher, and the fluidities of the respective resins also become higher. The conveying speed for the joined flexible tube material 131 is made constant, and the discharge amounts of the soft resin 139 and the hard resin 140 in a molten state are varied. In this manner, the thickness of each of the molded inner layer 117 and the molded outer layer 118 is adjusted.

The gates 135 and 136 are located outside the molding passage 137, with the molding passage 137 being the center of both gates. The gate 136 is located outside the gate 135. The gates 135 and 136 are cylindrical passages each having a circular cross-sectional shape in the direction perpendicular to the axial direction of the molding passage 137. The downstream ends of the gates 135 and 136 in the feeding direction of the soft resin 139 and the hard resin 140 connect to the upstream end of the resin passage 138. The connecting point is the meeting point where the soft resin 139 and the hard resin 140 join together. A separating portion 143 that separates the gates 135 and 136 from each other is provided between the gates 135 and 136.

The separating portion 143 has an edge 143a at the meeting point, and separates the gates 135 and 136 from each other on the upstream side of the meeting point. The soft resin 139 and the hard resin 140 fed through the respective gates 135 and 136 join together, immediately after passing through the edge 143a. To cause the two resins to join together, the edge 143a has a tapered cross-sectional shape that is narrower at the distal end in a direction parallel to the axial direction.

At the meeting point where the soft resin 139 and the hard resin 140 join together, the molten soft resin 139 fed through the gate 135 is located inside, and the molten hard resin 140 fed through the gate 136 is located outside. In this manner, the soft resin 139 and the hard resin 140 join together in an overlapping manner. As shown in FIGS. 9 and 10, the soft resin 139 and the hard resin 140 that have joined together flow in the resin passage 138 in an overlapped state. In FIGS. 9 and 10, reference numeral 145 designates the boundary between the soft resin 139 and the hard resin 140 in the resin passage 138. The soft resin 139 and the hard resin 140 in the overlapped state are discharged from the discharge outlet connecting to the whole circumference of the molding passage 137 in a circumferential direction, toward the whole circumference of the joined flexible tube material 131. In this manner, the outer coat layer 115 consisting of the two layers, which are the inner layer 117 and the outer layer 118, is molded.

The process that is carried out to form the outer coat layer 115 on the joined flexible tube material 131 with the continuous molding machine 120 having the above structure is now described. When the continuous molding machine 120 carries out molding procedures, the soft resin 139 and the hard resin 140 in a molten state are extruded from the extrusion units 121 and 122 to the head unit 123, and the conveyor unit 125 is activated to convey the joined flexible tube material 131 to the head unit 123.

At this point, the extrusion units 121 and 122 are in such a state as to constantly extrude and feed the soft resin 139 and the hard resin 140 to the head unit 123. The soft resin 139 and the hard resin 140 extruded from the extrusion units 121 and 122 into the gates 135 and 136 pass through the edge 143a and then join together. The soft resin 139 and the hard resin 140 in an overlapped state are fed into the molding passage 137 through the resin passage 138. In this manner, the outer coat layer 115 having a two-layer molded structure in which the inner layer 117 made of the soft resin 139 and the outer layer 118 made of the hard resin 140 overlap with each other is formed.

The joined flexible tube material 131 has flexible tube materials 114 joined to one another, and the outer coat layer 115 is formed continuously on the flexible tube materials 114 being conveyed in the molding passage 137. When the outer coat layer 115 is molded for one flexible tube material 114 extending from the one end 114a (the distal end) to the other end 114b (the proximal end), the control unit 126 controls the amounts of resins to be discharged from the extrusion units 121 and 122, so that the thickness ratio between the inner layer 117 and the outer layer 118 is 9:1 immediately after the start of the resin discharge from the extrusion units 121 and 122, the thickness ratio of the outer layer 118 gradually becomes higher in the intermediate portion between the one end 114a and the other end 114b of the flexible tube material 114, and the thickness ratio between the inner layer 117 and the outer layer 118 becomes 1:9 at the other end 114b of the flexible tube material 114.

Each joint member 130 is a joining part between two flexible tube materials 114, and therefore, the control unit 126 is used to change the amounts of resins to be discharged from the extrusion units 121 and 122. Specifically, the control unit 126 changes the amounts of resins to be discharged from the extrusion units 121 and 122, so that the thickness ratio at the other end 114b (the proximal end) of one flexible tube material 114 is changed to the thickness ratio at the one end 114a (the distal end) of the next flexible tube material 114.

When the outer coat layer 115 is molded for the next flexible tube material 114 extending from the one end 114a (the distal end) to the other end 114b (the proximal end), the extrusion units 121 and 122 are controlled so that the outer layer 118 also becomes thicker from the one end 114a toward the other end 114b. Thereafter, the same procedures as above are repeated, to form the outer coat layer 115 on the entire joined flexible tube material 131.

The joined flexible tube material 131 having the outer coat layer 115 formed even on the last end thereof is removed from the continuous molding machine 120. After that, the joint members 130 are removed from the joined flexible tube material 131, to divide the joined flexible tube material 131 into respective flexible tube materials 114. The coat film 116 is then formed on the outer coat layer 115 of each of the separated flexible tube materials 114, and the flexible tube 110 is completed. The completed flexible tube 110 is conveyed for the procedures for assembling the electronic endoscope 10.

As described above, the flexible tube 110 includes the outer coat layer 115 that has excellent molding precision and the hardness difference required between the distal end and the proximal end can be secured. In this embodiment, to obtain the above described outer coat layer 115, the materials of the inner layer 117 and the outer layer 118 are two different kinds of resins. Between the two different kinds of resins, the difference in 100% modulus value, which is the indicator of the hardness after molding, is 10 MPa or larger, and the difference in melt viscosity, which is the indicator of the fluidity of a resin in a molten state, is 2500 PaS or smaller at a molding temperature of 150 to 200° C.

Examples of combinations of resins that can satisfy the above two requirements include a combination of a resin selected from polyurethane-based resins and a resin selected from polyester-based resins. In this case, the soft resin 139 is selected from polyurethane-based resins, and the hard resin 140 is selected from polyester-based resins. Between a polyurethane-based resin and a polyester-based resin, the difference in 100% modulus value is large, and the difference in melt viscosity at a molding temperature of 150 to 200° C. is small.

A combination of resins that satisfy the above two requirements can also be selected from polyurethane-based resins. The resins are not limited to polyurethane-based resins and polyester-based resins, and a combination of resins that satisfy the above requirements can also be selected from synthetic resins such as polymer compounds.

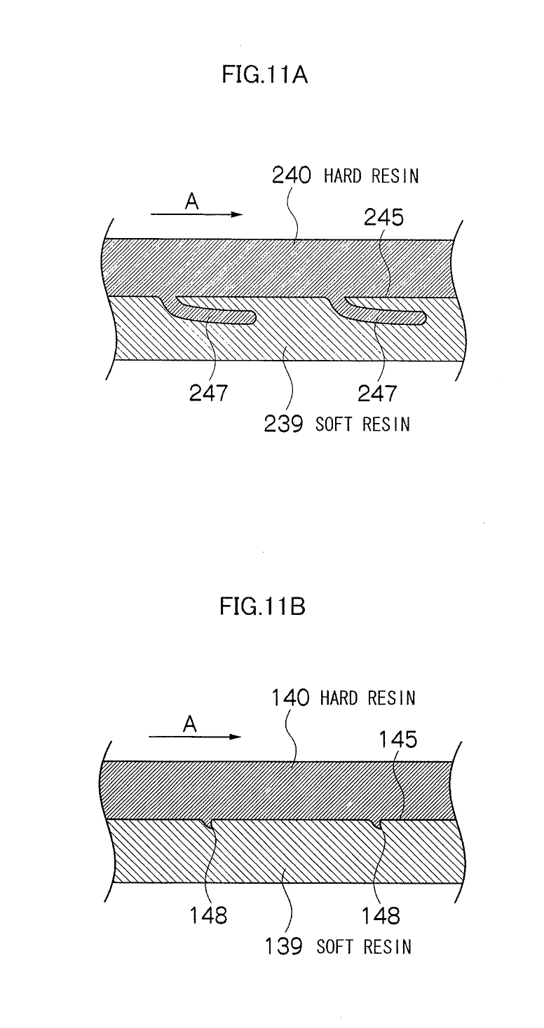

In the following, the difference in modulus value and the difference in melt viscosity at a molding temperature are described in detail. Referring to FIGS. 11A and 11B, the melt viscosity difference of 2500 PaS or smaller at the molding temperature of 150 to 200° C., which is the requirement for achieving excellent molding precision, is first described.

FIGS. 11A and 11B illustrate situations where a soft resin 239 and a hard resin 240 fed through the gates 135 and 136 join together at the meeting point, are heated to the molding temperature of 150 to 200° C., and flow from the resin passage 138 into the molding passage 137. FIG. 11A illustrates a comparative example in which the difference in melt viscosity between the soft resin 239 and the hard resin 240 at the molding temperature of 150 to 200° C. does not satisfy the above requirement (exceeding 2500 PaS). FIG. 11B illustrates this embodiment in which the difference in melt viscosity between the soft resin 139 and the hard resin 140 at the molding temperature of 150 to 200° C. satisfies the above requirement (equal to or smaller than 2500 PaS).

Specifically, the soft resin 239 and the hard resin 240 shown in FIG. 11A have a melt viscosity of 500 PaS and a melt viscosity of 6000 PaS, respectively, at the molding temperature of 150 to 200° C. The difference in melt viscosity is 5500 PaS. In this case, the soft resin 239 has a much lower melt viscosity (softer) than the hard resin 240. When there is a large difference in melt viscosity, the difference in flow rate between the soft resin 239 and the hard resin 240 is also large, and part of the hard resin 240 penetrates deep into the soft resin 239 in the vicinities of the boundary 245. Reference numeral 247 designates the penetrating portions of the hard resin 240 in the soft resin 239, and arrow A indicates the flowing direction of the hard resin 240.

The penetrating portions 247 are large, and large unevenness appears in the vicinities of the boundary 245. Therefore, the inner layer 117 and the outer layer 118 have uneven thicknesses in a circumferential direction, and cannot have desired thicknesses. Since the thickness of the outer coat layer 115 is approximately 0.2 to 1.0 mm, such penetrating portions 247 have large influence on the respective thicknesses of the inner layer 117 and the outer layer 118.

Meanwhile, the soft resin 139 and the hard resin 140 shown in FIG. 11B have a melt viscosity of 500 PaS and a melt viscosity of 3000 PaS, respectively, at the molding temperature of 150 to 200° C. This satisfies the requirement for the difference in melt viscosity to be equal to or smaller than 2500 PaS at the molding temperature of 150 to 200° C.

In the case of the soft resin 139 and the hard resin 140, the difference in melt viscosity and the difference in flow rate are small. Accordingly, the penetrating portions of the hard resin 140 in the soft resin 139 are small, as shown in FIG. 11B. Reference numeral 148 designates the penetrating portions of the hard resin 140 in the soft resin 139 around the boundary 145. The penetrating portions 148 cause small unevenness in the vicinity of the boundary 145, but the degree of penetration is so low that the penetrating portions 148 can be ignored in relation to the thickness of each layer. In view of this, excellent molding precision should be achieved by using the soft resin 139 and the hard resin 140 with a difference in melt viscosity of 2500 PaS or smaller at the molding temperature of 150 to 200° C.

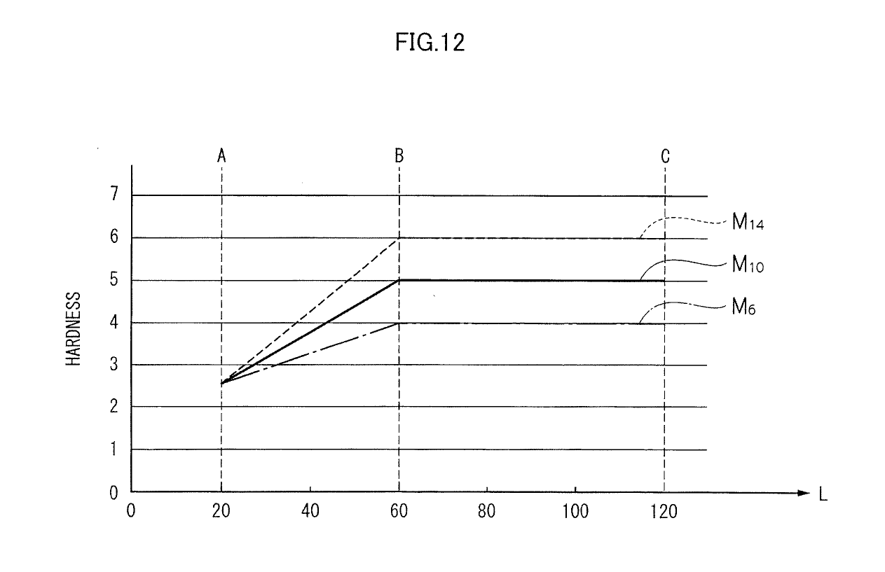

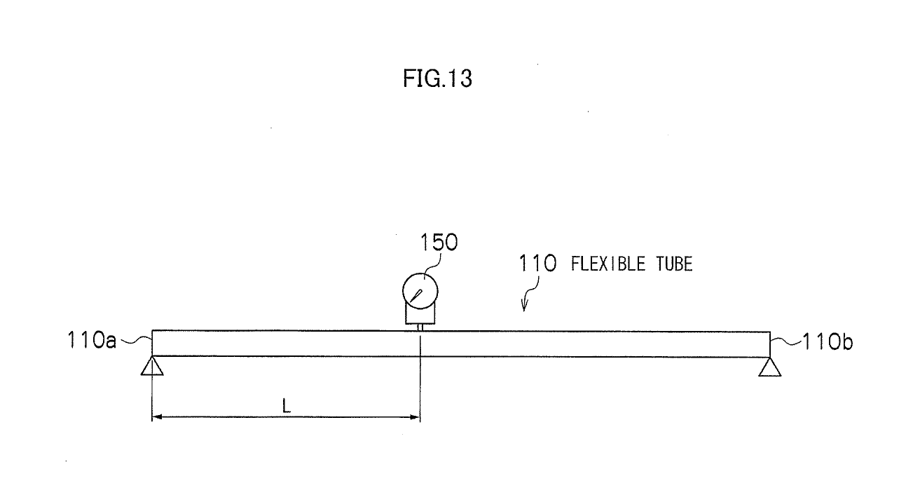

Referring now to FIGS. 12 and 13, the 100% modulus value difference of 10 MPa or larger is described as a requirement for obtaining the necessary hardness difference between the distal end and the proximal end of the outer coat layer 115. A modulus value represents the stress per unit area when a certain stretching force is applied to the subject material. The higher the modulus value is, the harder the material is. A 100% modulus value represents the stress per unit area (the stress applied in the stretching direction/the cross-sectional area perpendicular to the stretching direction) when the material is subjected to 100% stretching (that is, when the length of the material is made twice greater than that in the initial state).

FIG. 12 shows the results of measurement carried out to measure the hardness distributions of three kinds of two-layer molded flexible tubes with various difference in 100% modulus value between resins. In each of the hardness distributions, the measurement points where the hardness is measured are represented by the distance L (cm) from the distal end 110a of the flexible tube 110 (see FIG. 13).

The solid line M10 shows the hardness distribution obtained in a case where a soft resin of 2 MPa and a hard resin of 12 MPa are combined, and the difference in 100% modulus value is 10 MPa. The dotted line M14 shows the hardness distribution obtained in a case where a soft resin of 2 MPa and a hard resin of 16 MPa are combined, and the difference in 100% modulus value is 14 MPa. The solid line M10 and the dotted line M14 satisfy the requirement for the difference in 100% modulus value to be 10 MPa or larger.

On the other hand, the dot-and-dash line M6 shows the hardness distribution obtained in a comparative example in which a soft resin of 2 MPa and a hard resin of 8 MPa are combined, and the difference in 100% modulus value is 6 MPa, which is smaller than 10 MPa.

The three kinds of flexible tubes on which the hardness measurement was carried out are to be used in the insertion units of endoscopes for the large intestine, and each of the three flexible tubes has a total length of 130 cm. The outer diameters D are 11 to 14 mm, and the thicknesses of the outer coat layers 115 are 0.2 to 1.0 mm. In the zone between the distal end 110a and a point A of 20 cm (L=0 to 20 cm), each outer coat layer 115 is formed at the thickness ratio of 9:1 (the thickness of the inner layer 117:the thickness of the outer layer 118). In the zone between the point A and a point B of 40 cm (L=20 to 60 cm), the thickness of each outer layer 118 becomes gradually greater (the thickness of each inner layer 117 becomes gradually smaller). In the zone between the point B and the proximal end 110b (L=60 to 130 cm), each outer coat layer 115 is formed at the thickness ratio of 1:9 (the thickness of the inner layer 117:the thickness of the outer layer 118).

When the hardness of a flexible tube is measured, the flexible tube 110 is supported at both ends 110a and 110b, and the reaction forces that are generated by pressing the flexible tube 110 at respective measurement points in the axial direction are measured, as shown in FIG. 13. Reference numeral 150 in the drawing designates the hardness meter that measures the reaction forces. A larger reaction force indicates higher hardness at each point.

According to the solid line M10 showing the case where the difference in 100% modulus value is 10 MPa, the hardness in the vicinity of the hard proximal end 110b (a point C at L=120 cm) is twice higher than the hardness in the vicinity of the soft distal end 110a (the point A at L=20 cm).

Two different resins with a 100% modulus value difference of 10 MPa are used as the inner layer 117 and the outer layer 118, and the thickness ratio between the two resins is varied in the axial direction. In this manner, the hardness in the vicinity of the proximal end 110b can be made twice higher than the hardness in the vicinity of the distal end 110a.

As shown by the dotted line M14, in the case where the difference in 100% modulus value is 14 MPa, the hardness in the vicinity of the proximal end 110b (the point C at L=120 cm) is higher than twice the hardness (2.4 times higher than the hardness) in the vicinity of the distal end 110a (the point A at L=20 cm).

According to the dot-and-dash line M6 showing the case where the difference in 100% modulus value is 6 MPa as opposed to the cases shown by the solid line M10 and the dotted line M14, the hardness in the vicinity of the proximal end 110b (the point C at the distance L=120 cm) is lower than twice the hardness (1.6 times higher than the hardness) in the vicinity of the distal end 110a (the point A at L=20 cm).

To secure readiness in inserting the insertion unit 14, the hardness in the vicinity of the proximal end 110b needs to be at least twice higher than the hardness in the vicinity of the distal end 110a. Particularly, the insertion units 14 of lower gastrointestinal tract endoscopes for examining the large intestine need to satisfy this requirement. The large intestine has more curved portions such as the sigmoid colon having a small curvature radius than the upper gastrointestinal tracts such as the esophagus and the stomach. Therefore, a sophisticated technique is required in insertion when the large intestine is examined, and the endoscope is expected to have an insertion unit that is easier to be inserted, compared with an endoscope for the upper gastrointestinal tracts.

As shown by the solid line M10 and the dotted line M14, when the difference in 100% modulus value is 10 MPa or larger, a hardness difference equal to or larger than the required minimum hardness difference (twofold) between the distal end 110a and the proximal end 110b can be secured. On the other hand, as shown by the dot-and-dash line M6, the requisite hardness difference cannot be secured when the difference in 100% modulus value is smaller than 10 MPa.

In view of the above, as long as the difference in 100% modulus value is 10 MPa or larger, the required minimum hardness difference can be secured. In the example shown by the dotted line M14, the required minimum hardness difference can be secured even when the thickness ratio between the inner layer 117 and the outer layer 118 is made lower (1.5:8.5, for example) than the ratio mentioned above as an example. Therefore, the thickness ratio between the inner layer 117 and the outer layer 118 is not a requisite condition for securing the required minimum hardness difference, but can be changed with the difference in 100% modulus value as needed.

As described above, the inner layer 117 (the soft resin 139) and the outer layer 118 (the hard resin 140) as the two molded layers of the outer coat layer 115 are two different kinds of resins that satisfy the following two conditions: the difference in melt viscosity should be 2500 PaS or smaller at the molding temperature of 150 to 200° C., and the difference in 100% modulus value should be 10 MPa or larger. With this arrangement, excellent molding precision and the hardness difference required between the distal end and the proximal end can be both secured.

In the above described embodiment, the two-layer molded outer coat layer is formed by forming a soft resin layer as the inner layer and a hard resin layer as the outer layer. However, a hard resin layer may be formed as the inner layer, and a soft resin layer may be formed as the outer layer.

By using the above described two-layer molding, the resin layer forming the outer coat of the flexible tube forming the flexible portion 26 can include the two layers of a soft resin layer and a hard resin layer. The thickness ratio between the two layers is varied, so that the hardness distribution shown by the graph D in FIG. 5 can be obtained.

An endoscope and its flexible portion according to the present invention have been described in detail so far. However, the present invention is not limited to the above examples, and various changes and modifications may of course be made to those examples without departing from the scope of the invention.

What is claimed is:

1. An endoscope comprising:
an endoscope flexible portion that includes:
a low-hardness varying portion which is located in a predetermined region extending from a distal end of the flexible portion, and has hardness varying from the lowest hardness in the flexible portion to a predetermined hardness higher than the lowest hardness, the hardness being the lowest hardness at the distal end of the flexible portion, the hardness being the predetermined hardness at a proximal end of the low-hardness varying portion;
a hard portion which is located in a predetermined region extending from a proximal end of the flexible portion, and has the highest hardness in the flexible portion; and
an intermediate-hardness varying portion which is located between the low-hardness varying portion and the hard portion, and has hardness gradually varying in a region extending from the proximal end of the low-hardness varying portion to a distal end of the hard portion.

2. The endoscope according to claim 1, further comprising:
a hardness adjusting device which changes hardness of the flexible portion and is located in the flexible portion, the hardness adjusting device including: a hardness adjusting member which is capable of changing flexibility of the flexible portion; a hardness changing device which acts on the hardness adjusting member and changes hardness of the hardness adjusting member; and a drive device which drives the hardness changing device.

3. The endoscope according to claim 2, wherein a distal end portion of the hardness adjusting member is located in the low-hardness varying portion.

4. The endoscope according to claim 2, wherein a distal end portion of the hardness adjusting member is located in the intermediate-hardness varying portion.

5. The endoscope according to claim 2, wherein
the hardness adjusting member is a contact spring,
the hardness changing device is a wire which is inserted through the contact spring, and
the drive device is a wire pulling device which pulls the wire.

6. The endoscope according to claim 3, wherein
the hardness adjusting member is a contact spring,
the hardness changing device is a wire which is inserted through the contact spring, and
the drive device is a wire pulling device which pulls the wire.

7. The endoscope according to claim 4, wherein
the hardness adjusting member is a contact spring,
the hardness changing device is a wire which is inserted through the contact spring, and
the drive device is a wire pulling device which pulls the wire.

8. The endoscope according to claim 1, wherein
the variation in hardness of the low-hardness varying portion and the intermediate-hardness varying portion of the flexible portion is formed by causing a gradient in hardness of a resin layer forming an outer coat of a flexible tube forming the flexible portion.

9. The endoscope according to claim 2, wherein
the variation in hardness of the low-hardness varying portion and the intermediate-hardness varying portion of the flexible portion is formed by causing a gradient in hardness of a resin layer forming an outer coat of a flexible tube forming the flexible portion.

10. The endoscope according to claim 3, wherein
the variation in hardness of the low-hardness varying portion and the intermediate-hardness varying portion of the flexible portion is formed by causing a gradient in hardness of a resin layer forming an outer coat of a flexible tube forming the flexible portion.

11. The endoscope according to claim 4, wherein
the variation in hardness of the low-hardness varying portion and the intermediate-hardness varying portion of the flexible portion is formed by causing a gradient in hardness of a resin layer forming an outer coat of a flexible tube forming the flexible portion.

12. The endoscope according to claim 5, wherein
the variation in hardness of the low-hardness varying portion and the intermediate-hardness varying portion of the flexible portion is formed by causing a gradient in hardness of a resin layer forming an outer coat of a flexible tube forming the flexible portion.

13. The endoscope according to claim 6, wherein
the variation in hardness of the low-hardness varying portion and the intermediate-hardness varying portion of the flexible portion is formed by causing a gradient in hardness of a resin layer forming an outer coat of a flexible tube forming the flexible portion.

14. The endoscope according to claim 7, wherein
the variation in hardness of the low-hardness varying portion and the intermediate-hardness varying portion of the flexible portion is formed by causing a gradient in hardness of a resin layer forming an outer coat of a flexible tube forming the flexible portion.

15. The endoscope according to claim 8, wherein the hardness gradient in the resin layer is caused through two-layer molding performed by varying a thickness ratio between a soft resin and a hard resin.

16. A flexible portion of an endoscope, comprising:
a low-hardness varying portion which is located in a predetermined region extending from a distal end of the flexible portion, and has hardness varying from the lowest hardness in the flexible portion to a predetermined hardness higher than the lowest hardness;
a hard portion which is located in a predetermined region extending from a proximal end of the flexible portion, and has the highest hardness in the flexible portion; and
an intermediate-hardness varying portion which is located between the low-hardness varying portion and the hard portion, and has hardness gradually varying in a region extending from the proximal end of the low-hardness varying portion to a distal end of the hard portion.

17. The flexible portion according to claim 16, further comprising:
a hardness adjusting device which includes: a contact spring which has a distal end thereof located in the low-hardness varying portion, and is capable of changing flexibility of the flexible portion; and a wire which is pulled and relaxed to act on the contact spring and change hardness of the contact spring.

18. The flexible portion according to claim 16, further comprising:
a hardness adjusting device which includes: a contact spring which has a distal end thereof located in the intermediate-hardness varying portion, and is capable of changing flexibility of the flexible portion; and a wire which is pulled and relaxed to act on the contact spring and change hardness of the contact spring.
